(19) United States
(12) Patent Application Publication
(10) Pub. No.: US 2014/0318552 A1
(43) Pub. Date: Oct. 30, 2014

(54) SPINAL IMMOBILIZATION DEVICE

(71) Applicant: Jonathan Pinto, San Diego, CA (US)

(72) Inventor: Jonathan Pinto, San Diego, CA (US)

(21) Appl. No.: 14/261,410

(22) Filed: Apr. 24, 2014

Related U.S. Application Data
(60) Provisional application No. 61/815,595, filed on Apr. 24, 2013.

(51) Int. Cl. A61G 1/048; A61B 6/04; A61G 1/044

(52) U.S. Cl. CPC A61G 1/048; A61G 1/044; A61B 6/0428
USPC 128/876; 128/870

(57) ABSTRACT

A spinal immobilization device is disclosed. The spinal immobilization device may include a plurality of cleats disposed on the bottom of the spinal immobilization device to prevent damage to the spinal immobilization device. The spinal immobilization device may include one or more pieces of reflective tape disposed on the perimeter of the transparent board frame to illuminate the spinal immobilization device and to protect the one or more caregiver users and the second user. The spinal immobilization device may accommodate one or more ambidextrous users, where either side of the spinal immobilization device may be utilized as a head side or a foot side. The spinal immobilization device may be made of transparent material and X-Ray translucent material to allow the one or more caregiver users to easily view the second user's injuries and allow X-raying the second user's injuries while the second user is secured in the spinal immobilization device.

This application claims priority to U.S. Provisional Application 61/815,595 filed on Apr. 24, 2014, the entire disclosure of which is incorporated by reference.

### FIELD OF THE INVENTION

The present invention generally relates to a device. More specifically, the invention is a spinal immobilization device.

### DESCRIPTION OF THE RELATED ART

Several millions of people in the United States are required to be placed in full cervical spinal immobilization devices every year by first responder personnel. Some of the injuries that require full “C-Spine” are those who have a trauma incident, such us a car accident that provides a traumatic shock to the body. These traumatic events may potentially harm their spinal cord, further causing them to become paraplegic or quadriplegic in severe cases. Other patients that may require this are those with multiple gunshots or stabbing to the trunk region of the body.

### BRIEF SUMMARY OF THE INVENTION

The present invention generally relates to a device. More specifically, the invention is a spinal immobilization device.

The spinal immobilization device includes a transparent board frame having a perimeter, a head end and a foot end, one or more first users grasp the transparent board frame from the head end and the foot end by a plurality of cut-out handles disposed on the perimeter of the transparent board, the transparent board frame allowing one or more first users to view a second user and a second user's one or more injuries and the board is secured within the transparent board frame by a plurality of tabs securing the board within the transparent board frame, the one or more first users grasping the transparent board frame from the head end and the foot end, wherein the spinal immobilization device accommodates one or more ambidextrous users, where either side of the spinal immobilization device may be utilized as a head side or a foot side. The spinal immobilization device includes one or more fasteners coupled to the perimeter of the transparent board, the one or more fasteners secure the second user to the transparent board, the board is made of X-Ray translucent material to allow X-raying the second user's injuries while the second user is secured in the spinal immobilization device and a plurality of cleats disposed on the bottom of the transparent board, the cleats secure the transparent board frame in one or more positions and to protect the transparent board. The spinal immobilization device also includes one or more pieces of reflective tape disposed on the perimeter of the transparent board frame to illuminate the spinal immobilization device and to protect the one or more first users and the second user.

It is an object of the present invention to provide a spinal immobilization device that is made of transparent material and X-Ray translucent material to allow rescue worker users to easily view a patient user's injuries and allow X-raying those injuries while the patient user is secured in the spinal immobilization device.

It is an object of the present invention to provide a spinal immobilization device that includes a plurality of cleats disposed on the bottom of the spinal immobilization device to prevent damage to the spinal immobilization device.

It is an object of the present invention to provide a spinal immobilization device that includes a reflective tape lining disposed around a perimeter of the spinal immobilization device which will allow for further road side protection of rescue personnel users.

It is an object of the present invention to provide a spinal immobilization device that accommodates one or more ambidextrous users, where either side of the spinal immobilization device may be utilized as a head side or a foot side.

### BRIEF DESCRIPTION OF THE DRAWINGS

The present invention will be described by way of exemplary embodiments but not limitations, illustrated in the accompanying drawings in which like references denote similar elements, and in which:

FIG. 1 illustrates an overhead view of a spinal immobilization device, in accordance with one embodiment of the present invention.

FIG. 2 illustrates an overhead view of a transparent board frame, in accordance with one embodiment of the present invention.

FIG. 3 illustrates a side view of a spinal immobilization device in a folded position, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention however the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

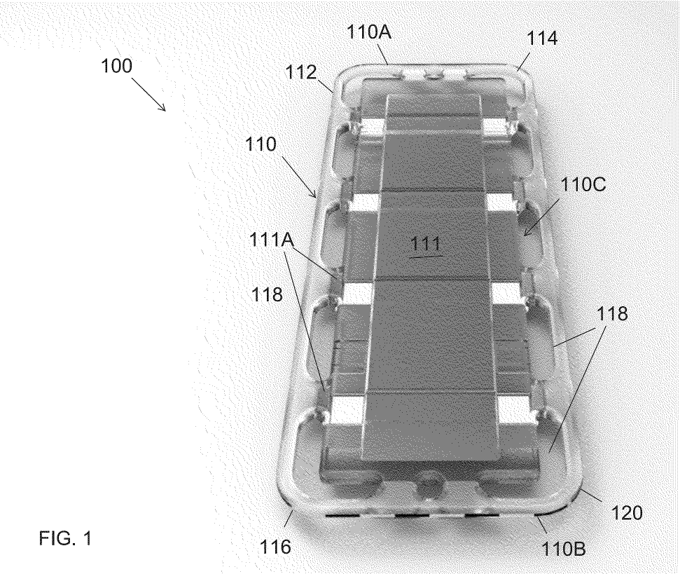

FIG. 1 illustrates an overhead view of a spinal immobilization device 100, in accordance with one embodiment of the present invention.

The spinal immobilization device 100 may include a transparent board frame 110 and one or more pieces of reflective tape 120. The transparent board frame 110 may include a perimeter 112, a head end 114 and a foot end 116. The transparent board frame 110 may include a board 111 disposed within an indentation 110C of the transparent board frame 110. The board 111 may be secured within the transparent board frame 110 by a plurality of tabs 111A securing the board 111 within the transparent board frame 110. The transparent board frame 110 may allow one or more first users to view a second user and a second user's one or more injuries. The one or more first users may be a caregiver user or other suitable user. The second user may be a patient user or other suitable user. The one or more first users may grasp the transparent board frame 110 from a head end 110A and a foot end 110B by a plurality of cut-out handles 118 disposed on the perimeter 112 of the transparent board frame 110. The spinal immobilization device 100 may accommodate one or more ambidextrous users, where either side of the spinal immobilization device may be utilized as the head end 110A or the foot end 110B. The one or more pieces of reflective tape 120 may be disposed on the perimeter 112 of the transparent board frame 110 to illuminate the spinal immobilization device 100 and to protect the one or more first users and the second user.

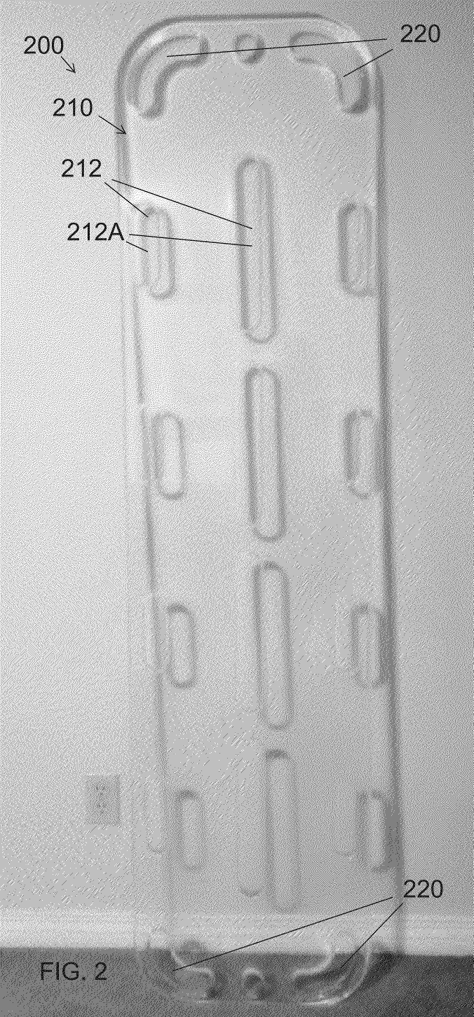

FIG. 2 illustrates an overhead view of a transparent board frame 200, in accordance with one embodiment of the present invention.

The transparent board frame 200 may include a rear facing 210 and a plurality of cleats 220. The rear facing 210 may include a plurality of apertures 212 disposed on the rear facing 210 that may allow one or more caregiver users to access one or more injuries of a patient user. The apertures 212 may be a plurality of elongated oval apertures 212A disposed throughout the rear facing 210. The cleats 220 may be disposed on each bottom corner 210A of the rear facing 210. The cleats 220 may secure the transparent board frame 200 in one or more positions and protect the transparent board frame 200 from damage.

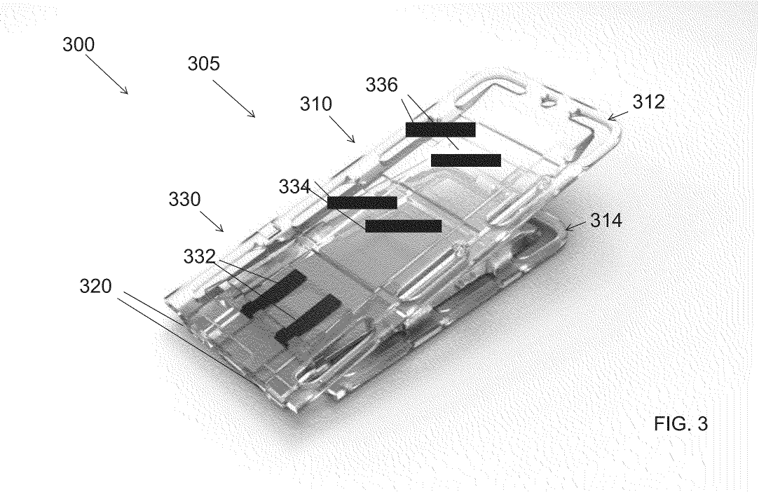

FIG. 3 illustrates a side view of a spinal immobilization device 300 in a folded position 305, in accordance with one embodiment of the present invention.

The spinal immobilization device 300 may include a transparent board frame 310, one or more hinges 320 and one or more fasteners 330. The transparent board frame 310 may include a first half 312 and a second half 314. The transparent board frame 310 is illustrated in FIG. 3 without the board 111. The one or more hinges 320 may be coupled to the first half 312 and the second half 314 of the transparent board frame 310 and allow the first half 312 and the second half 314 of the transparent board frame 310 to fold outward or fold inward. The one or more fasteners 330 may be coupled to the transparent board frame 310. The one or more fasteners 330 may secure the second user to the transparent board frame 310. The one or more fasteners 330 may be selected from the group consisting of one or more spider straps 332, one or more buckled straps 334 and one or more pieces of duct tape 336.

The spinal immobilization device will allow for a full back view of a patient user's spine once a patient user has already been in place in full C-spine precautions (i.e., spinal precautions). This allows caregiver users to quickly treat an injury that has been missed or has been delayed to be discovered due to scene chaos or rapid immobilization. The spinal immobilization device will include many of the features that come with the other current backboards which include being X-ray translucent, easy to utilize, user friendly and easy to clean. The spinal immobilization device also will be made of transparent material and X-Ray translucent material. The spinal immobilization device also includes a plurality of cleats disposed on the bottom of the board to prevent damage to the spinal immobilization device. The spinal immobilization device also includes a reflective tape lining which will allow for further road side protection of rescue personnel users. Rescue personnel users will be able to utilize the spinal immobilization device uniformly. The head of the spinal immobilization device may go towards either end of the spinal immobilization device without worry about being “foot first” or “head first” which may save time and is more user-friendly.

The spinal immobilization device may accommodate one or more ambidextrous users, where either side may be utilized as a head side or a foot side. There is also a reflective tape lining which will allow for further road side protection of the rescue personnel. The spinal immobilization device also includes a plurality of cleats on the bottom of the spinal immobilization device that protect the transparent material of the spinal immobilization device.

The spinal immobilization device is utilized as a transparent back board. The user patients will be placed on the backboard with their underside exposed for trauma. The user patients will then be placed into full cervical spine immobilization precautions to prevent further back injuries or disabilities from current injuries.

The spinal immobilization device is a temporary field body splint to protect a patient user's body from further insult to a patient's user's spine and create an environment of stability so the patient user does not progress to a devastating injury. The spinal immobilization device allows rescue personnel users to have an unobstructed view of an injury during an entire cervical spine immobilization process. The spinal immobilization device is utilized when a patient user has multiple gunshot injuries or multiple stab injuries. Once rescue personnel users place the patient user in a full immobilization position on one or more backboards, they limit the areas that they may see to potentially protect the patient user from further injury or even death. This is due to the way the traditional backboards are made and utilized.

The spinal immobilization device is designed so rescuers may find a serious non-conspicuous injury causing a patient user to be in critical condition until they are rolled off the spinal immobilization device. This may very well be a significant reason the patient user dies or bleeds out. The spinal immobilization device is utilized by users that do not have a way to see the patient user's back clearly once the injured patient user is on the spinal immobilization device.

The spinal immobilization device may help locate one or more missed exit injuries that may result in a patient user becoming rapidly unstable. The spinal immobilization device allows one or more rescue personnel users to have a clear way to look for a backside injury or insult without having to compromise spine immobilization procedures. The spinal immobilization device allows a paramedic, emergency medical technician or EMT, Corpsmen and other rescue personnel to see an underside (i.e., spinal area, back of the legs, back of femur area, back of head, and back of shoulders) of a patient user's body.

The spinal immobilization device may save lives as it has been proven that a patient user may bleed out from the blood in their femurs. The spinal immobilization device provides a better chance of finding an insult or injury to see the major components of the backside of a patient user's body.

While the present invention has been related in terms of the foregoing embodiments, those skilled in the art will recognize that the invention is not limited to the embodiments described. The present invention may be practiced with modification and alteration within the spirit and scope of the appended claims. Thus, the description is to be regarded as illustrative instead of restrictive on the present invention.

What is claimed is:

1. A spinal immobilization device, comprising:
a transparent board frame having a perimeter and a board, the transparent board frame allowing one or more first users to view a second user and a second user's one or more injuries and the board is secured within the transparent board frame by a plurality of tabs securing the board within the transparent board frame; and
one or more fasteners coupled to the perimeter of the transparent board, the one or more fasteners secure the second user to the transparent board.

2. The spinal immobilization device according to claim 1, wherein the one or more first users is a caregiver user.

3. The spinal immobilization device according to claim 1, wherein the second user is a patient user.

4. The spinal immobilization device according to claim 1, wherein the board is made of X-Ray translucent material to allow X-raying the second user's injuries while the second user is secured in the spinal immobilization device.

5. The spinal immobilization device according to claim 1, wherein the one or more fasteners are one or more spider straps.

6. The spinal immobilization device according to claim 1, wherein the one or more fasteners are one or more buckled straps.

7. The spinal immobilization device according to claim 1, wherein the one or more fasteners are one or more pieces of duct tape.

8. A spinal immobilization device, comprising:
a transparent board frame having a perimeter, a head end and a foot end, one or more caregiver users grasp the transparent board frame from the head end and the foot end by a plurality of cut-out handles disposed on the perimeter of the transparent board, the transparent board frame allowing the one or more caregiver users to view a patient user and a patient user's one or more injuries and the board is secured within the transparent board frame by a plurality of tabs securing the board within the transparent board frame; and
one or more fasteners coupled to the perimeter of the transparent board, the one or more fasteners secure the patient user to the transparent board.

9. The spinal immobilization device according to claim 8, further comprising a plurality of cleats disposed on the bottom of the transparent board, the cleats secure the transparent board frame in one or more positions and to protect the transparent board.

10. The spinal immobilization device according to claim 8, wherein the transparent board frame allowing one or more caregiver users to view a patient user and the one or more injuries, the one or more caregiver users grasping the transparent board frame from the head end or the foot end.

11. The spinal immobilization device according to claim 8, wherein the board is made of X-Ray translucent material to allow X-raying the patient user's one or more injuries while the patient user is secured in the spinal immobilization device.

12. The spinal immobilization device according to claim 8, wherein the one or more fasteners are one or more spider straps.

13. The spinal immobilization device according to claim 8, wherein the one or more fasteners are one or more buckled straps.

14. The spinal immobilization device according to claim 8, wherein the one or more fasteners are one or more pieces of duct tape.

15. A spinal immobilization device, comprising:
a transparent board frame having a perimeter, a head end and a foot end, one or more first users grasp the transparent board frame from the head end and the foot end by a plurality of cut-out handles disposed on the perimeter of the transparent board, the transparent board frame allowing one or more first users to view a second user and a second user's one or more injuries and the board is secured within the transparent board frame by a plurality of tabs securing the board within the transparent board frame, the one or more first users grasping the transparent board frame from the head end and the foot end, wherein the spinal immobilization device accommodates one or more ambidextrous users, where either side of the spinal immobilization device may be utilized as a head side or a foot side;
one or more fasteners coupled to the perimeter of the transparent board, the one or more fasteners secure the second user to the transparent board, the board is made of X-Ray translucent material to allow X-raying the second user's injuries while the second user is secured in the spinal immobilization device;
a plurality of cleats disposed on the bottom of the transparent board, the cleats secure the transparent board frame in one or more positions and to protect the transparent board; and
one or more pieces of reflective tape disposed on the perimeter of the transparent board frame to illuminate the spinal immobilization device and to protect the one or more first users and the second user.

16. The spinal immobilization device according to claim 15, wherein the one or more first users is a caregiver user.

17. The spinal immobilization device according to claim 15, wherein the second user is a patient user.

18. The spinal immobilization device according to claim 15, wherein the one or more fasteners are selected from the group consisting of one or more spider straps, one or more buckled straps and one or more pieces of duct tape.

19. The spinal immobilization device according to claim 15, wherein the transparent board frame is foldable.

20. The spinal immobilization device according to claim 19, wherein the spinal immobilization device is a cervical spinal immobilization device.
